(19) United States
(12) Patent Application Publication
(10) Pub. No.: US 2014/0180257 A1
(43) Pub. Date: Jun. 26, 2014

(54) COVER FOR AN ATRAUMATIC CATHETER HUB AND A METHOD FOR ITS USE

(71) Applicant: Kenneth M. Zinn, Westport, CT (US)

(72) Inventor: Kenneth M. Zinn, Westport, CT (US)

(21) Appl. No.: 13/724,617

(22) Filed: Dec. 21, 2012

(51) Int. Cl. A61M 39/16

(52) U.S. Cl. CPC A61M 39/165
USPC 604/533

(57) ABSTRACT

An atraumatic cover for a catheter hub. The cover comprises an opening configured to allow a line/tube to pass through the opening when the cover is in a closed configuration. The cover can comprise one or more pliable inserts located within the cover. In an embodiment, the pliable insert can comprise an antibacterial agent. The atraumatic cover for a catheter hub can be formed in any number of different shapes and/or sizes.

### FIELD OF THE INVENTION

The present general inventive concept is directed toward an atraumatic cover for a catheter hub and a method for its use.

### BACKGROUND

Central venous catheters, peripherally inserted central catheters (PICCs), hemodialysis catheters, and other similar devices comprise hubs at their ends. Hubs are threaded plastic connections located at the ends of a catheter apparatus that are located outside of a patient's body. Hub assemblies often comprise clamps located adjacent to the ends of such hubs, which are used to control the flow of fluid through the lines or tubing to which the hub is connected. These hubs and clamps are typically located at the ends of a short length of line/tube, which protrude from the patient's skin.

Three problems commonly arise relating to the hubs and clamps that comprise such catheters. First, these hubs and clamps can have sharp edges and points, which can cause discomfort for the patient when they contact the patient's skin. Second, these hubs and clamps can be accidentally manipulated or snagged on clothing, which can be harmful to both the patient and the catheter apparatus. Third, these devices can become contaminated by dirt or bacteria, which can increase the risk of infection.

In an attempt to address these three problems, gauze is sometimes wrapped around these hubs and clamps and secured in place with tape. However, this solution can be unsightly, easily dislodged and time-consuming. Furthermore, there is no antibacterial feature of standard gauze or tape, which could help prevent bacterial contamination of the hub or clamp to occur.

What is needed is an atraumatic catheter hub cover, which can protect a patient's skin from sharp edges comprising the hub and clamp, prevent the hub and clamp from being accidentally manipulated or snagged, and prevent the hub from becoming contaminated by dirt or bacteria.

### SUMMARY OF THE INVENTION

It is an aspect of the present device to provide an improved atraumatic catheter hub cover.

The above can be obtained by an atraumatic catheter hub cover, comprising: a first half comprising a first pliable insert and a first notch located at one end of the first half; a second half comprising a second pliable insert and a second notch located at one end of the second half; wherein the first half is connected to the second half so that the first half and second half are configured to be moved between a closed configuration and an open configuration; and when the first half and the second half are moved into the closed configuration, the first notch and the second notch are configured to align to form an opening configured to allow a tube to pass through the opening.

The above aspect can also be obtained by an atraumatic catheter hub cover comprising: a cylinder, comprising a first end and a second end, wherein the first end is closed and the second end is open; and a door is connected to the second end by a hinge, wherein the door comprises a notch, and the notch is configured to create a first opening when placed in a closed configuration wherein the first opening is configured to allow a tube to pass through the first opening.

The above aspect can also be obtained by an atraumatic catheter hub cover comprising: a squeezable foam comprising a first end and a second end opposite the first end, wherein the first end is closed and the second end is open; and an elongated opening at the second end further comprising a first opening end and a second opening end; wherein the squeezable foam is configured such that at rest the hub cover is in a closed configuration and when pressure is applied to the first opening end and the second opening end the hub cover opens into an open configuration.

These together with other aspects and advantages, which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Further features and advantages of the present inventive concept, as well as the structure and operation of its various embodiments, will become apparent and more readily appreciated from the following description of the preferred embodiments, taken in conjunction with the accompanying drawings of which:

FIG. 1 is a partial front view of a prior art catheter apparatus comprising a hub and a clamp connected to a tube/line;

FIG. 2 is a top and side perspective view of an egg-shaped hub cover in an open configuration, according to a first embodiment;

FIG. 2A is a front view of the egg-shaped hub cover in an open configuration, and a (prior art) hub and clamp comprising a hub assembly, according to the first embodiment;

FIG. 2B is a front, cutaway view of the egg-shaped hub cover in a closed configuration, wherein a (prior art) hub and clamp has been enclosed within the egg-shaped hub cover, according to the first embodiment;

FIG. 2C is a bottom view of the egg-shaped hub cover in a closed configuration, according to the first embodiment;

FIG. 2D is a front view of the egg-shaped hub cover in a closed configuration according to the first embodiment;

FIG. 3 is a bottom and side perspective view of a rectangular hub cover in an open configuration, according to a second embodiment;

FIG. 4 is a front view of the rectangular hub cover in a closed configuration, according to the second embodiment;

FIG. 5 is a bottom view of the rectangular hub cover in a closed configuration, according to the second embodiment;

FIG. 6 is a top view of the rectangular hub cover in a closed configuration, according to the second embodiment;

FIG. 7 is a top, side and bottom perspective view of the rectangular hub cover in a closed configuration, wherein a (prior art) hub and clamp (not visible) are located within the hub cover and a (prior art) tube/line passes from the hub (not visible), through an opening in the hub cover, according to the second embodiment;

FIG. 8 is a front view of a catheter apparatus comprising a first hub and a first clamp comprising a catheter apparatus and a rectangular hub cover enclosing a second hub (not visible) and a second clamp (not visible), according to the second embodiment;

FIG. 9 is a bottom and side perspective view of a cylindrical hub cover comprising a door in an open configuration, according to a third embodiment;

FIG. 9A is a bottom and side transparent view of a cylindrical hub cover comprising a door in a closed configuration, wherein a (prior art) hub and clamp are located within the cylindrical hub cover and a (prior art) tube/line is shown passing from the (prior art) hub through notch in the door, according to a third embodiment;

FIG. 9B is a bottom and side perspective view of a cylindrical hub cover comprising a door in a closed configuration, wherein a (prior art) hub and clamp (not visible) are located within the cylindrical hub cover and a (prior art) tube/line is shown passing from the (prior art) hub through notch in the door, according to a third embodiment;

FIG. 9C is a bottom view of a cylindrical hub cover comprising a door in a closed configuration, wherein a (prior art) hub and clamp (not visible) are located within the cylindrical hub cover and a (prior art) tube/line is shown passing from the (prior art) hub through notch in the door, according to a third embodiment;

FIG. 10 is a top and side perspective view of a squeezable hub cover, according to a fourth embodiment;

FIG. 10A is a bottom view of a squeezable hub cover in an open configuration, wherein (prior art) two hubs and clamps (not visible) are located within the squeezable hub cover and (prior art) two tubes/lines are shown passing from the (prior art) hub (not visible) through an opening at the bottom of the squeezable hub cover, according to a fourth embodiment; and

FIG. 10B is a bottom view of a squeezable hub cover in a closed configuration, wherein (prior art) two hubs and clamps (not visible) are located within the squeezable hub cover and (prior art) two tubes/lines are shown passing from the (prior art) hub (not visible) through an opening at the bottom of the squeezable hub cover, according to a fourth embodiment.

### DESCRIPTION OF THE PREFERRED EMBODIMENTS

Reference will now be made in detail to the presently preferred embodiments of the present inventive concept, examples of which are illustrated in the accompanying drawings, wherein like reference numerals refer to like elements throughout.

The present inventive concept relates to an atraumatic cover for a catheter hub which can protect a patient's skin from sharp edges comprising the hub and clamp, prevent the hub and clamp from being accidentally manipulated or snagged, and prevent the hub from becoming contaminated by dirt or bacteria.

In particular, the present inventive concept relates to multiple embodiments of covers that that can be configured to contain one or more hubs that are connected to one or more catheters/lines/tubes. The present hub cover can comprise or consist of a foam or similar soft, pliable material that can be configured to receive a hub, clamp, and tubing connected to the hub or can be configured to mold to the shape of a hub, a clamp, and tubing connected to the hub. In an embodiment, this pliable material can comprise an antibacterial agent that is part of this material or has been applied to it. Any type of material or foam can be used for any part of any embodiment described herein. For example polyurethane, high density foam, Evlon®, high resilience foam, latex rubber foam, Supreem®, Rebond®, memory foam, closed cell foam, and dry fast foam or any combination of these materials can be used to construct the pliable material. In some embodiments, the pliable material can be located within a hard cover or case that can surround the pliable material. In other embodiments, an outer surface of the pliable material can be coated with a polymer or similar material that can create a sealing cover to the pliable material, thus preventing moisture, dirt, or other contaminants from reaching the inside of the pliable material or the hubs, clamps or tubing located within it.

The present inventive concept has numerous embodiments, each being formed in a different shape, including: an egg-shape, spherical-shape, cylindrical-shape, or rectangular-shape, wherein the outer surface of the device comprises only blunt or smooth edges, which can protect a patient from the sharp edges of a catheter hub or hub and clamp assembly.

An aspect of the present device is that it can be used to cover a hub and/or clamp that is connected to a line/tube comprising a catheter apparatus. In order to accommodate this tube/line, the cover can comprise one or more openings wherein the hub and clamp assembly can be inserted into the cover and one or more tube/line can extend from the cover to the patient. In an embodiment, each hub and clamp can be protected by one cover, or two or more hubs and clamps can be protected by a single cover.

In an embodiment, the device can comprise a slit configured to receive one or more hubs and/or clamps. This slit can be in a closed configuration when no pressure is applied to the sides of the cover. In the closed configuration, the hub and/or clamp can be secured within the cover and the tube/line can extend through the slit. In an embodiment, the slit can be placed in an open configuration by applying pressure to the sides of the cover opposite the slit. This open configuration can facilitate insertion of the catheter hub and clamp assembly into the cover.

In an embodiment, the cover device can comprise a first half and a second half. Each half can comprise an inner space and an outer surface, and a first end and a second end. In an embodiment, the inner space of each half can comprise a foam insert configured to receive a hub, clamp and tubing. In an embodiment, the first half and second half can be pivotably connected by a hinge allowing the first half and second half to be opened to a distance sufficient to allow the hub, clamp and tubing to be placed within the cover. In an embodiment, the second ends of one or both of each half can comprise an opening configured to allow the tube/line to pass from a hub located within the cover device, through the opening, and outside of the cover device. In an embodiment, the first half and the second half can snap together or otherwise become securely connected together in a closed configuration in order to secure hubs, clamps and tubing within these cover devices.

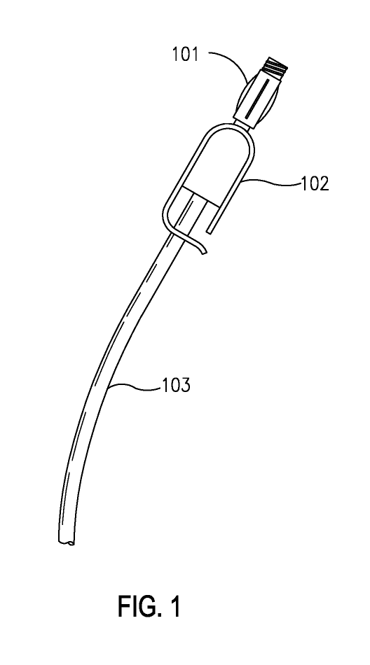

FIG. 1 is a partial front view of a prior art catheter apparatus 100 comprising a hub 101 and a clamp 102 connected to a tube/line 103.

Catheters and similar devices allow fluids to be injected into a patient's body and/or be removed from a patient's body through one or more tubes/lines 103. The hub 101 is the point of connection to the tube/line 103 and a syringe (not shown), dialysis machine (not shown) or other device that is used to push fluids into the body or pull fluids from the body. A clamp 102 is commonly used to prevent fluid, namely blood from passing from the patient through the hub 101 or to control the rate at which fluid can flow through the tube/line in either direction.

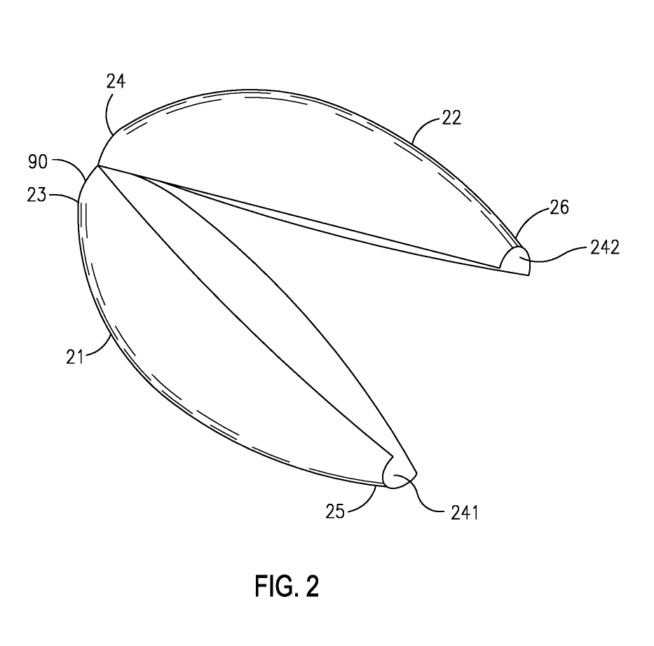

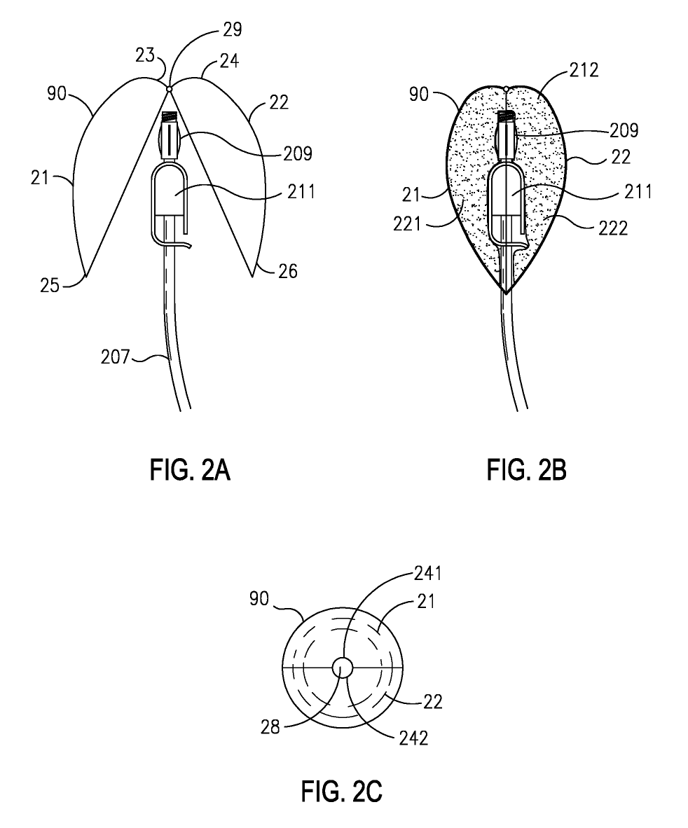

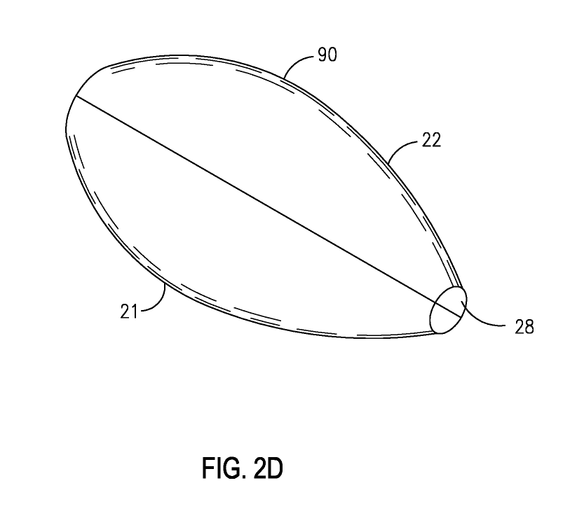

FIG. 2 is a top and side perspective view of an egg-shaped hub cover 90 in an open configuration, according to a first embodiment. The cover 90 can be egg-shaped in order to eliminate points or sharp edges that can poke or scratch the patient's skin or snag or catch clothing or other objects that the cover can contact. This shape can be configured to comprise an inner volume sufficient to contain at least one hub, clamp and a line/tube connected to the hub (not shown). In this embodiment, the egg-shaped hub cover 90 can comprise a first half 21 comprising a first end 23 and a second end 25 and a second half 22 comprising a first end 24 and a second end 26. In an embodiment, the first end 23 of the first half 21 and the first end 24 of the second half 22 can be pivotably connected by a hinge (not shown in FIG. 2). In a different embodiment, the first half 21 and the second half 22 can be configured to be connected by snapping or otherwise connecting the first half 21 to the second half 22 without the use of a hinge. The second end 25 of the first half 21 can comprise a first notch 241 and the second end 26 of the second half 22 can comprise a second notch 242. The first notch 241 and the second notch 242 can be aligned in a closed position (not shown) to create an opening (not shown) to allow the line/tube (not shown) to pass out of the egg-shaped hub cover 90.

FIG. 2A is a front view of the egg-shaped hub cover 90 in an open configuration, and a hub 209 and clamp 211 comprising a hub assembly 207 (prior art), according to the first embodiment.

FIG. 2A depicts a first half 21 and a second half 22 connected by a hinge 29 located at the first end 23 of the first half 21 and the first end 24 of the second half 22. In this view, the hinge 29 is located at the top of the egg-shaped hub cover 90 and the opposite end is located at the bottom of the egg-shaped hub cover 90. However, the hinge 29 could be located a different locations along the first half 21 and second half 22. Alternatively, the first half 21 and second half 22 could be configured to be snapped together, or could comprise an adhesive or other connecting feature sufficient to connect the first half 21 to the second half 22.

FIG. 2B is a front, cutaway view of the egg-shaped hub cover 90 in a closed configuration, wherein a (prior art) hub 209 and clamp 211 have been enclosed within the egg-shaped hub cover 90, according to the first embodiment.

In this view, the inner volume 212 can be viewed as well as the position of the hub 209 and clamp 211 within the inner volume. In an embodiment, first half 21 can comprise a first section 221 of pliable material and the second half 22 can comprise a second section 222 of pliable material, wherein the hub 209 and clamp 211 can be secured between the first section 221 of pliable material and the second section 222 of pliable material. In an embodiment, the pliable material can be a foam or any other similar suitable material.

FIG. 2C is a bottom view of the egg-shaped hub cover 90 in a closed configuration, according to the first embodiment.

FIG. 2C shows how an opening 28 configured to allow a tube/line (not shown) to pass through it, can be formed by a first notch 241 on a bottom of the first half 21 and a second notch 242 on a bottom of the second half 22. Both notches 241 and 242 align to form opening 28.

FIG. 2D is a side view of the egg-shaped hub cover 90 in a closed configuration, and a (prior art) hub and clamp comprising a hub assembly, according to the first embodiment.

This view also shows how the first half 21 and the second half 22 of the egg-shaped hub cover 90 can align to form an opening 28.

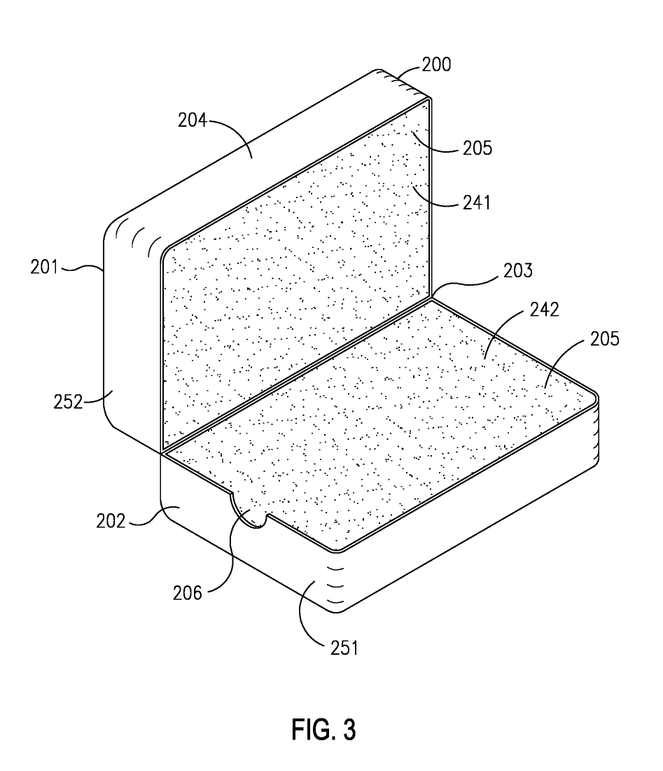

FIG. 3 is a bottom and side perspective view of a rectangular hub cover 200 in an open configuration, according to a second embodiment.

In this second embodiment, the rectangular hub cover 200 can comprise a first half 201 and a second half 202. The first half 201 and the second half 202 can be connected by a hinge 203 allowing the hub cover 200 to be moved from an open configuration as shown or a closed configuration wherein a hub and clamp (not shown), or similar object can be securely held within the hub cover 200. In an embodiment, a first notch 206 or similar opening can be located at a first end 251 of the second half 202. In an alternative embodiment, a second notch (not shown) can be located at the first end 252 of the first half 201 and the first notch 206 can align to form an opening, through which a line/tube can pass from the hub cover 200 to a patient (not shown).

In an embodiment, the first half 201 and the second half 202 can be comprised of a hard outer surface 204, such as a plastic or similar material. A pliable material 205, such as a foam or any other suitable material, can occupy the first inner volume 241 of the first half 201 and second inner volume 242 of the second half 202. This pliable material 205 can comprise cavities (not shown) that are shaped to receive one or more hubs or clamps (not shown) or the pliable material can also be configured to conform to the shape of various objects that can been enclosed within the hub cover 200. In an embodiment, the pliable material 205 can also comprise an antibacterial agent, such as triclosan or any other suitable antibacterial agent known in the art. Said antibacterial/antimicrobial agent will be compatible with and not damage the underlying catheter hub apparatus.

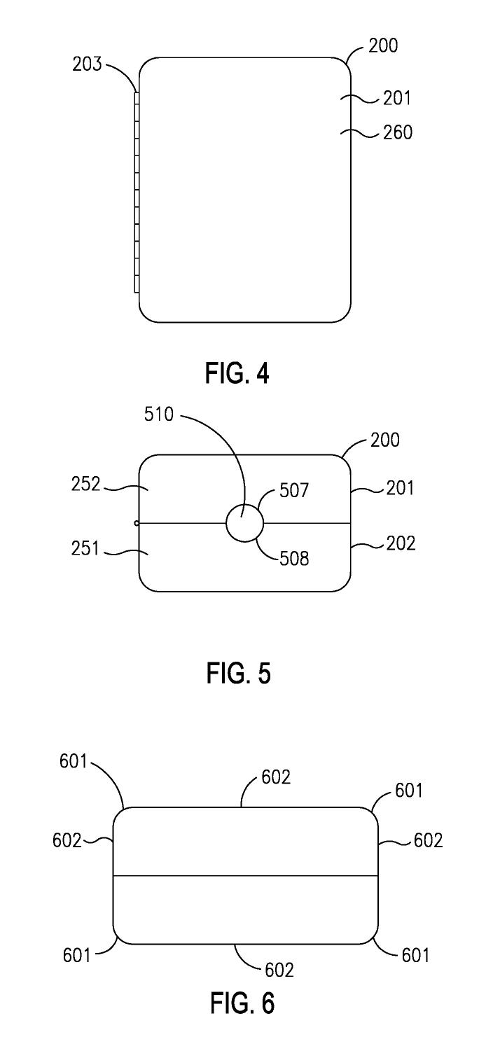

FIG. 4 is a front view of the rectangular hub cover 200 in a closed configuration, according to the second embodiment.

This view shows the outer surface 260 of the first half 201 of the hub cover 200 and shows the hinge 203 from outside the hub cover 200.

FIG. 5 is a bottom view of the rectangular hub cover 200 in a closed configuration, according to the second embodiment.

This view shows the opening 510, which can be configured to allow one or more tubes/lines to pass through the hub cover 200 when placed in a closed configuration. In this embodiment the opening 510 is created by a first notch 507 located at the first end 251 of the first half 201 and a second notch 508 located at the second end 252 of the second half 202. In an embodiment, the first notch 507 and the second notch 508 can align to create an opening 510 configured to allow the tube/line to pass through it.

FIG. 6 is a top view of the rectangular hub cover 200 in a closed configuration, according to the second embodiment.

In order to solve two of the problems associated with the uncovered hubs, namely patient discomfort and snagging of pointed and sharp edges, the corners 601 and edges 602 of the hub cover 200 can be blunt, smooth, or curved.

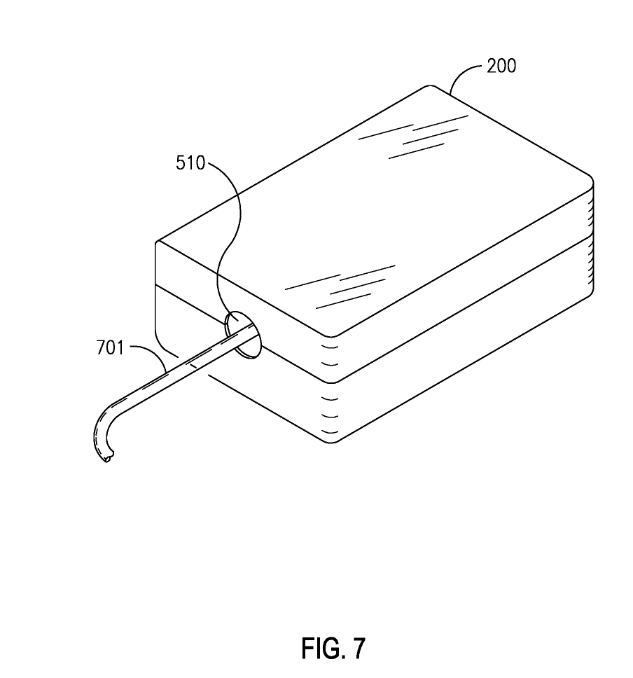

FIG. 7 is a top, side and bottom perspective view of the rectangular hub cover 200 in a closed configuration, wherein a (prior art) hub and clamp (not visible) are located within the hub cover and a (prior art) tube/line 701 passes from the hub (not visible), through an opening 510 in the hub cover 200, according to the second embodiment

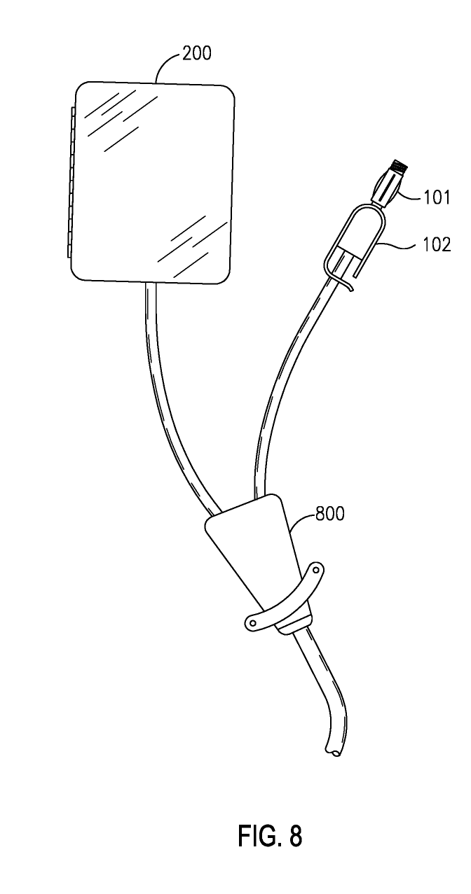

FIG. 8 is a front view drawing of a catheter apparatus 800 comprising a first hub 101 and a first clamp 102 comprising a catheter apparatus 800 and a rectangular hub cover 200 enclosing a second hub (not visible) and a second clamp (not visible), according to the second embodiment.

This view demonstrates how an uncovered hub 101 and clamp 102 would appear and contrast that to a hub (not visible) and clamp (not visible) enclosed within a hub cover 200.

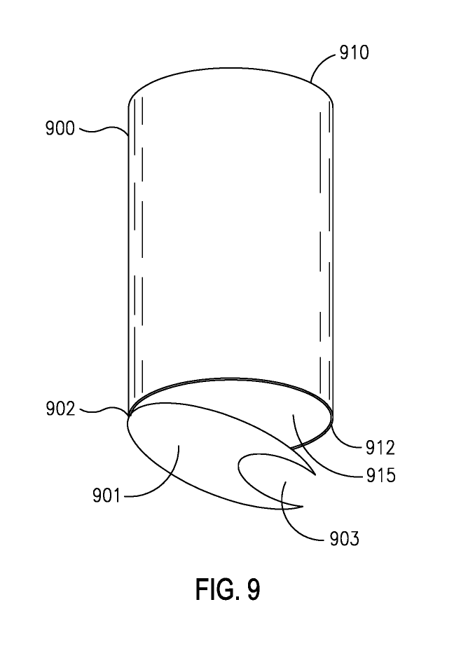

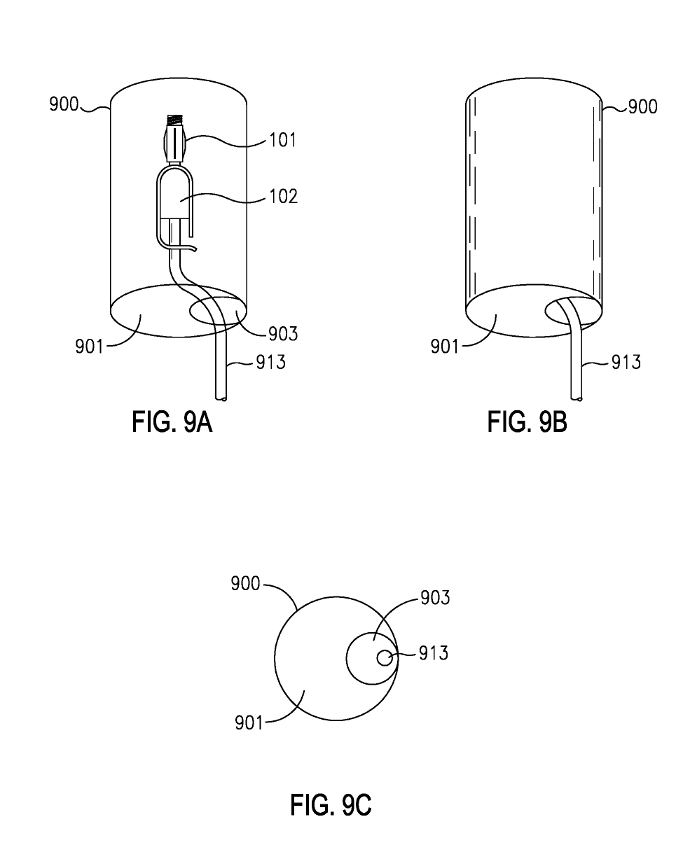

FIG. 9 is a bottom and side perspective view of a cylindrical hub cover 900 comprising a door 901 in an open configuration, according to a third embodiment.

In this third embodiment, the device can be a cylindrical hub cover 900 being closed at a first end 910 and comprising a door 901 located at a second end 912. The cylindrical hub cover 900 can have an inner volume 915 configured to contain at least one hub and clamp (not shown). In an embodiment, the cylindrical hub cover 900 can be lined with a pliable material (not visible in FIG. 9) to protect the hub. In an embodiment, the door 901 can be connected to the second end 912 by a hinge 902 and can comprise an opening 903 configured to allow a tube/line (not shown) to pass through it.

FIG. 9A is a bottom and side transparent view of a cylindrical hub cover 900 comprising a door 901 in a closed configuration, wherein a hub (prior art) 101 and clamp (prior art) 102 are shown in FIG. 9A, but would ordinarily not be visible through the hub cover 900. These are located within the cylindrical hub cover 900 and a tube/line (prior art) 913 is shown passing from the hub (prior art) 101 through the opening 903 in the door 901, according to a third embodiment.

FIG. 9B is a bottom and side perspective view of a cylindrical hub cover 900 comprising a door 901 in a closed configuration, wherein a (prior art) hub (not visible) and clamp (not visible) are located within the cylindrical hub cover 900 and a (prior art) tube/line 913 is shown passing from the (prior art) hub through opening 903 in the door 901, according to a third embodiment.

FIG. 9C is a bottom view of a cylindrical hub cover 900 comprising a door 901 in a closed configuration, wherein a (prior art) hub and clamp (not visible) are located within the cylindrical hub cover 900 and a (prior art) tube/line 913 is shown passing through opening 903 in the door 901, according to a third embodiment;

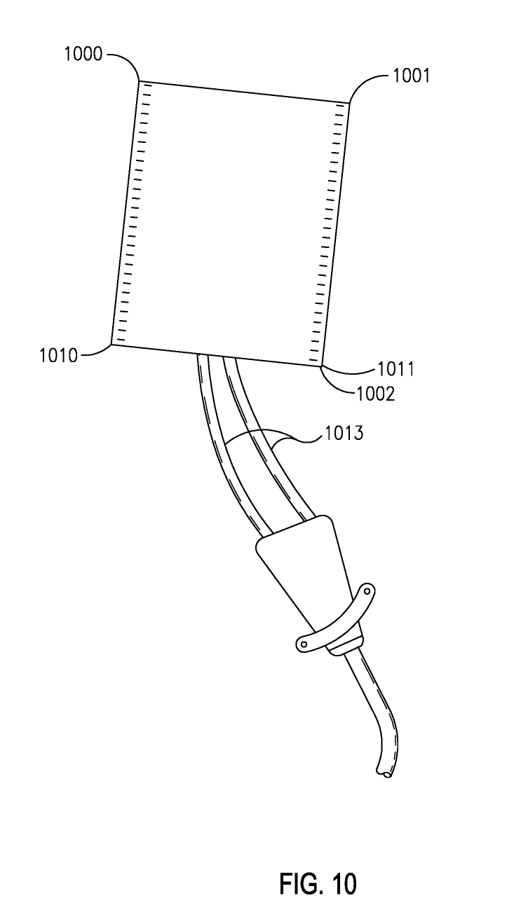

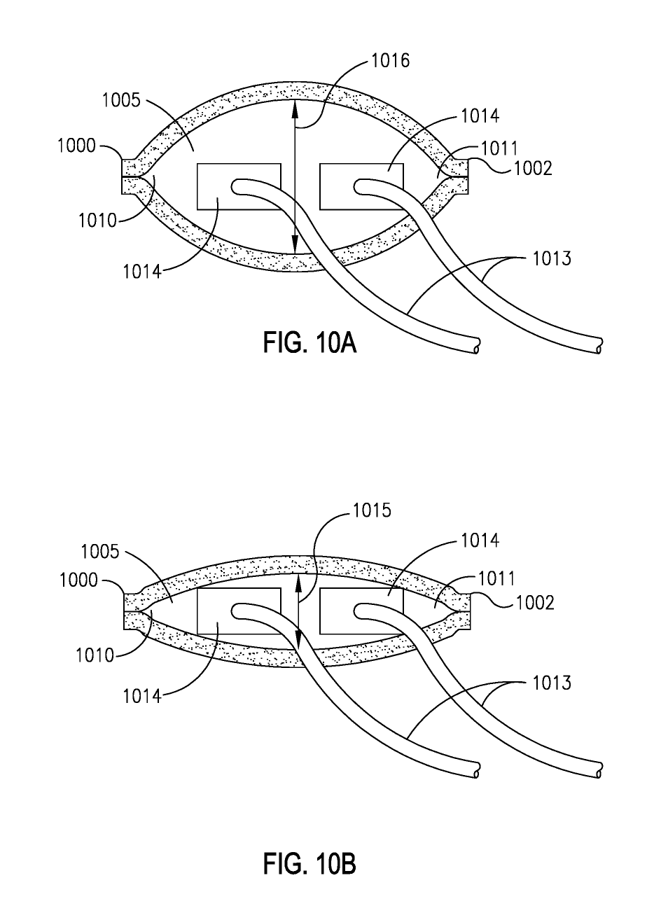

FIG. 10 is a top and side perspective view of a squeezable hub cover 1000, according to a fourth embodiment.

In this fourth embodiment, a squeezable hub cover 1000 can be substantially comprised of a flexible foam, rubber, or similar suitable material. In an embodiment, the squeezable hub cover 1000 can be closed at a first end 1001 and comprise an opening (not visible in FIG. 10) of a first width at a second end 1002, when the squeezable hub cover 1000 is placed in a closed position. In this fourth embodiment, at least one hub and clamp (not visible in FIG. 10) can be placed within the squeezable hub cover 1000 while in an open position and the hub and clamp can be retained within the hub cover 1000 while in a closed position. A hub cover and clamp can quickly be inserted into the hub cover 1000 by squeezing the first opening end 1010 and the second opening end 1011 together and placing the hub and clamp within the hub cover 1000 while it is in an open position, then releasing the first opening end 1010 and the second opening end 1011 and allowing the hub cover 1000 to retain the hub and clamp while in a closed position.

FIG. 10A is a bottom view of the second end of a squeezable hub cover 1000 in an open configuration, wherein two hubs and clamps (prior art) 1014 are located within the squeezable hub cover 1000 and two tubes/lines (prior art) 1013 are shown passing from the hub (prior art) (not visible) through an opening 1005 at the bottom of the squeezable hub cover 1000.

The opening 1005 can have an elongated shape comprising a first opening end 1010 and a second opening end 1011, wherein the opening 1005 can be a second width 1016, greater than the first width 1015 as shown in FIG. 10B, when the first opening end 1010 and second opening end 1011 are pressed together and the squeezable hub cover 1000 can be in an open position.

In this view, a second width 1016 and its relation to the size of the clamps 1014 can be viewed. The second width 1016 is the width when the squeezable hub cover 1000 is in the open configuration and the opening should be sufficiently wide to allow one or more hubs (not visible) and clamps 1014 to be placed within the squeezable hub cover 1000.

FIG. 10B is a bottom view of the second end of a squeezable hub cover 1000 in a closed configuration, wherein (prior art) two hubs and clamps 1014 are located within the squeezable hub cover 1000 and (prior art) two tubes/lines 1013 are shown passing from the (prior art) hub (not visible) through an opening 1005 at the bottom of the squeezable hub cover 1000, according to a fourth embodiment.

In this view, the first width 1015 (when the squeezable hub cover 1000 is in the closed configuration) and its relation to the size of the clamps 1014 can be viewed as there are clamps 1014 inside the squeezable hub cover 1000 and the first width 1015 is not zero but is still wide enough to provide room for the contents inside the hub cover 1000. The first width 1015 should be sufficient to secure one or more hubs (not visible) and clamps 1014 within the squeezable hub cover 1000 so that they cannot be easily removed without first squeezing the first opening end 1010 and the second opening end 1011 together and thus placing the squeezable hub cover 1000 in an open configuration as shown in FIG. 10A. The squeezable hub cover 1000 can be made of a pliable material such as foam, etc., or any material described herein. The material is such that it can be easily deformed by squeezing the sides (which puts the cover 1000 in the open configuration) but has shape retaining properties such that when the pressure is removed (the sides are no longer squeezed) the hub cover 1000 will quickly and automatically revert to the closed configuration as shown in FIG. 10B. In an embodiment, the squeezable hub cover 1000 can comprise a moisture resistant outer surface.

All parts herein can be made of the standard materials used for such objects known in the art.

The many features and advantages of the inventive concept are apparent from the detailed specification and, thus, it is intended by the appended claims to cover all such features and advantages of the concept that fall within its true spirit and scope. Further, since numerous modifications and changes will readily occur to those skilled in the art, it is not desired to limit the inventive concept to the exact construction and operation illustrated and described, and accordingly all suitable modifications and equivalents may be resorted to, falling within the scope of the inventive concept.

What is claimed is:

1. An atraumatic catheter hub cover, comprising:
a first half comprising a first pliable insert and a first notch located at one end of the first half;
a second half comprising a second pliable insert and a second notch located at one end of the second half;
wherein the first half is connected to the second half so that the first half and second half are configured to be moved between a closed configuration and an open configuration; and
when the first half and the second half are moved into the closed configuration, the first notch and the second notch are configured to align to form an opening configured to allow a tube to pass through the opening.

2. The apparatus as recited in claim 1 wherein the first half and the second half are connected by a hinge.

3. The apparatus as recited in claim 1 wherein the first pliable insert and the second pliable insert comprise an antibacterial agent.

4. The apparatus as recited in claim 1 wherein the first pliable insert has a cavity in a shape of a hub.

5. The apparatus as recited in claim 1 wherein the first half and the second half when in the closed position form a substantially rectangular box-shaped container.

6. The apparatus as recited in claim 1 wherein the first half and the second half when in the closed position form a substantially egg-shaped container.

7. An atraumatic catheter hub cover comprising:
a cylinder, comprising a first end and a second end, wherein the first end is closed and the second end is open; and
a door is connected to the second end by a hinge, wherein the door comprises a notch, and the notch is configured to create a first opening when placed in a closed configuration wherein the first opening is configured to allow a tube to pass through the first opening.

8. The apparatus as recited in claim 7 wherein an inner surface of the cylinder is lined with a pliable material.

9. The apparatus as recited in claim 8 wherein the pliable material comprises an antibacterial agent.

10. An atraumatic catheter hub cover comprising:
a squeezable foam comprising a first end and a second end opposite the first end, wherein the first end is closed and the second end is open; and
an elongated opening at the second end further comprising a first opening end and a second opening end;
wherein the squeezable foam is configured such that at rest the hub cover is in a closed configuration and when pressure is applied to the first opening end and the second opening end the hub cover opens into an open configuration.

11. An apparatus as described in claim 10 wherein the squeezable foam has a moisture resistant outer surface.

12. The apparatus as recited in claim 10, wherein the squeezable foam comprises an antibacterial agent.

13. A method for using an atraumatic hub cover, the method comprising:
providing an atraumatic catheter hub cover, comprising:
a first half comprising a first pliable insert and a first notch located at one end of the first half;
a second half comprising a second pliable insert and a second notch located at one end of the second half;
wherein the first half is connected to the second half so that the first half and second half are configured to be moved between a closed configuration and an open configuration; and
wherein when the first half and the second half are moved into the closed configuration, the first notch and the second notch are configured to align to form an opening configured to allow a tube to pass through the opening; and
providing a catheter comprising a hub connected to a line/tube; and
placing the atraumatic catheter hub cover in the open configuration;
placing the hub within the atraumatic catheter hub cover and positioning the line/tube so that it passes through the opening; and
placing the atraumatic catheter hub cover in a closed configuration.

14. A method for using an atraumatic hub cover, the method comprising:
providing an atraumatic catheter hub cover, comprising:
a cylinder, comprising a first end and a second end, wherein the first end is closed and the second end is open; and
a door is connected to the second end by a hinge, wherein the door comprises a notch, and the notch is configured to create a first opening when placed in a closed configuration wherein the first opening is configured to allow a tube to pass through the first opening; and
providing a catheter comprising a hub connected to a line/tube; and
opening the door of the atraumatic catheter hub cover;
placing the hub within the atraumatic catheter hub cover and positioning the line/tube so that it will pass through the first opening; and
placing the atraumatic catheter hub cover into a closed configuration.

15. A method for using an atraumatic hub cover, the method comprising:
providing an atraumatic catheter hub cover, comprising:
a squeezable foam comprising a first end and a second end opposite the first end, wherein the first end is closed and the second end is open; and
an elongated opening at the second end further comprising a first opening end and a second opening end;
wherein the squeezable foam is configured such that at rest the hub cover is in a closed configuration and when pressure is applied to the first opening end and the second opening end the hub cover opens into an open configuration; and
providing a catheter comprising a hub connected to a line/tube; and
placing the atraumatic catheter hub cover in an open configuration by squeezing the first opening end and the second opening end together;
placing the hub within the atraumatic catheter hub cover and positioning the line/tube so that it will pass through the first opening; and
placing the atraumatic catheter hub cover in a closed configuration by releasing the first opening end and the second opening end.
